(19) United States
(12) Patent Application Publication
(10) Pub. No.: US 2013/0143231 A1
(43) Pub. Date: Jun. 6, 2013

(54) MST1/STK4 PHOSPHO-THREONINE 120 (pMST-T120) ANTIBODY

(71) Applicants: Bekir Cinar, Los Angeles, CA (US); Filiz Kisaayak Collak, Los Angeles, CA (US)

(72) Inventors: Bekir Cinar, Los Angeles, CA (US); Filiz Kisaayak Collak, Los Angeles, CA (US)

(73) Assignee: CEDARS-SINAI MEDICAL CENTER, Los Angeles, CA (US)

(21) Appl. No.: 13/816,868

(22) PCT Filed: Aug. 19, 2011
(86) PCT No.: PCT/US11/48508

§ 371 Date: Feb. 13, 2013

Related U.S. Application Data
(60) Provisional application No. 61/375,425, filed on Aug. 20, 2010.

(51) Int. Cl. G01N 33/68; C07K 16/40

(52) U.S. Cl. CPC G01N 33/6893; C07K 16/40
USPC 435/7.1; 530/387.9

(57) ABSTRACT

The present invention relates to prostate cancer (PCa). More specifically, the invention provides a MST1 phosphothreonine (pMST1-T120) antibody that can be used in various assays to study correlations between Mst1 function and T120 site phosphorylation. The present invention can also be used to determine disease development and/or progression of prostate cancer in a subject using the antibodies disclosed herein.

### CROSS-REFERENCE TO RELATED APPLICATIONS

This application claims priority from U.S. Provisional Patent Application No. 61/375,425, filed on Aug. 20, 2010, which is incorporated herein by reference in its entirety.

### FIELD OF THE INVENTION

This invention relates to prostate cancer (PCa), other cancers and non-cancer tissues, in which pMst1 is expressed. More specifically, the invention provides an antibody and methods of use thereof for the detection of phosphorylated MST1 form in cells or tissues, including prostate cancer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

MST1/STK4 is a serine-threonine protein kinase, which was originally identified as a pro-apoptotic protein. MST1 is a 56-60 kDa protein that can be activated by autophosphorylation of a unique threonine residue (Thr-183) or by caspase-3 cleavage as a response to overexpression and a wide range of cell death stimuli, including chemotherapeutic agents such as bortezomib, a specific proteosome inhibitor. Accumulation of caspase-cleaved MST1 kinase (the catalytically active 34-36 kDa MST1-N form) in the nucleus has been proposed to induce apoptosis in cells via chromatin condensation. The inventors' studies indicate, however, that MST1 can reside in the nuclei of prostate cancer (PCa) cells, though cells remain normal. This suggests the possibility that nuclear-localized MST1 is functionally inactivated or has a novel function. Loss of MST1 function has been linked to the emergence of metastatic cancers and shown to correlate with chemoresistance in various cancer types. A published study from the inventors' laboratory suggests that MST1 may play a prominent role in PCa progression; however, the biological role of MST1 in PCa remains to be investigated.

PCa is the most commonly diagnosed cancer among men and the second leading cause of cancer-related death in the United States. Evidence suggesting the prevailing hypothesis is that cooperative androgen receptor (AR) and PI3K/PTEN/Akt-mTOR pathway signaling are critical to human prostate tumor development and progression to the metastatic state. Up to now, despite extensive studies, no effective treatment is offered for patients with metastatic PCa due to the lack of understanding the disease mechanism. Another problem in PCa management is the lack of molecular predictors, which can be used to stratify the risk of disease progression.

Previously, the inventors engineered inducible MST1 expression PCa cell models, in which MST1 can be expressed under the control of an inducible promoter that can be modulated with Doxycyclin, a tetracycline analog, to study the physiological role of the MST1 signal in PCa. The studies indicated that (i) induction of MST1 expression antagonizes AR activity and AR-mediated gene expression, whose portion product regulates PCa cell survival signals and metastasis, and (ii) suppresses the growth of human PCa cells in culture and tumor growth in mice. These observations indicate that the MST1 signal is a potent tumor suppressor and/or metastasis suppressor in PCa. An implication from these studies supports the notion that loss of MST1 function may be associated with the aggressive form of PCa.

### SUMMARY OF THE INVENTION

In certain embodiments, the invention teaches an isolated antibody that specifically binds to MST1 phospho-T120.

In some embodiments, the invention teaches a composition comprising an antibody that specifically binds to MST phospho-T120.

In various embodiments, the invention teaches a method of detecting MST1 phospho-T120 in a sample, including: providing a sample; contacting the sample with an antibody that binds to MST1 phospho-T120; allowing the antibody to bind to MST1 phospho-T120 in the sample; and detecting the presence of MST1 phospho-T120 in the sample. In some embodiments, the sample is obtained from a tumor. In some embodiments, the detection is accomplished by immunohystochemical analysis.

In certain embodiments, the invention teaches a method of determining late stage cancer in a subject, including: providing a sample of cells from a subject; contacting the sample from the subject with an antibody that binds to MST1 phospho-T120; allowing the antibody to bind to MST1 phospho-T120 in the sample; and detecting the presence of MST1 phospho-T120 in the nucleus of the cells and not in the cytoplasm of the cells, wherein detection of MST1 phospho-T120 in the nucleus of the cells and not in the cytoplasm indicates late stage cancer. In some embodiments, the sample is obtained from a tumor. In some embodiments, the detection is accomplished using immunohystochemical analysis. In some embodiments, the cancer is prostate cancer.

In various embodiments, the invention teaches a method of determining the amount of MST1 phospho-T120 in the cells of a sample, including: providing a sample of cells from a subject; contacting the sample with an antibody that binds to MST1 phospho-T120; and determining the amount of MST1 phospho-T120 in the cells of the sample. In some embodiments, the sample is obtained from a tumor.

In certain embodiments, the amount of MST1 phospho-T120 is determined using immunohystochemical analysis.

In certain embodiments, the invention teaches a method of determining early stage cancer in a subject including: providing a sample of cells from a subject; contacting the sample with an antibody that binds to MST1 phospho-T120; allowing the antibody to bind to MST1 phospho-T120 in sample; detecting the presence of MST1 phospho-T120 in the cytoplasm of the cells and in the nucleus of the cells, wherein detection of MST1 phospho-T120 in the cytoplasm of the cells and in the nucleus of the cells indicates early stage cancer in the subject.

In various embodiments, the invention teaches a method of determining the invasiveness of prostate cancer (PCa) in a subject, by determining that a modification is present in the MST1 protein of the prostate cancer cells, compared to the MST1 protein of non-cancerous cells.

In various embodiments, the invention teaches a method of diagnosing prostate cancer in a subject including: providing a sample of cells from a subject; contacting the sample from the subject with an antibody that binds to MST1 phospho-T120; allowing the antibody to bind to MST1 phospho-T120 of the sample; and detecting the presence of MST1 phospho-T120 in the cells of the sample, wherein detection of MST1 phospho-T120 in the cytoplasm of the cells and in the nucleus of the cells or only in the nucleus of the cells indicates prostate cancer. In some embodiments, the sample is obtained from a tumor. In certain embodiments, the detection is accomplished by immunohystochemical analysis.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 demonstrates, in accordance with an embodiment of the invention, the assessment of MST1 phospho-T120 levels in cells and in tissue section of human PCa. (A) Determination of phospho-MST1 T120 site specificity by immunoprecipitation (IP) and western blot (WB) analyses of total lysates from HEK293 cells expressing transient empty vector (Vec), wild type (wt) or T120A mutant Myc-tagged MST1. IP with anti-Myc antibody, WB with anti-phospho T120 or anti-Myc antibody were performed. (B-C) Western blot analyses of phospho-MST1 T120 (pMST1-T120) levels in total cell lysates from normal (PrePEC) or Pca (LNCaP or C4-2B) cells (B) and of pMST1-T120 levels in cytoplasmic (Cyt) and nuclear (Nuc) fractions from LNCaP cells that were grown in serum-depleted conditions (C). (D) Immunohystochemical staining of pMST1-T120 in tissue section from normal prostate and from the early or late stage PCa. Data are representative of multiple experiments.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

The present invention relates to the functional correlation between loss of Mst1 function and T120 site phosphorylation and its correlation and/or association with disease development or progression in PCa. The inventors have produced an antibody that specifically recognizes the MST1 phospho-T120 present in the nuclei of PCa cells. MST1 is a potential therapeutic target and prognostic biomarker in PCa.

Accordingly, one aspect of the present invention is directed to an isolated MST1 phospho-threonine (pMST1-T120) antibody. The antibody is polyclonal and specifically binds to the MST1 phospho-T120 peptide. The antibody can be used to determine the loss of Mst1 function and T120 site phosphorylation by specifically recognizing MST1 phospho-T120 in the nuclei of PCa cells and in tissue sections. The antibody can be used in western blots and immunohistochemistry (fixed and paraffin-embedded tissue section) assays. The antibody can also be used in immunoprecipitation, immunocytochemistry, and ELISA experiments.

A further aspect of the present invention provides a method of using the antibody of the present invention for detection, diagnosis, staging, and progression of PCa.

This invention also relates to the detection of MST1 phospho-T120 in other cancers and non-cancer tissues, and generally wherever pMst1 is expressed. More specifically, the invention provides an antibody and methods of use thereof for the detection of phosphorylated MST1 in any cells or tissues, including prostate cancer. The antibody of the present invention could be used for detection purposes in vivo or in vitro.

A peptide, such as CSDIIRLRNK{pThr}LTEDEIA (Sequence ID. No: 1) used to immunize an animal can be derived from translated cDNA or chemical synthesis and is purified and conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal antibodies can be further purified, for example, by binding to and eluting from a matrix to which a polypeptide or a peptide to which the antibodies were raised is bound. Those of skill in the art will know of various techniques common in the immunology arts for purification and/or concentration of polyclonal antibodies, as well as monoclonal antibodies. (See, for example, Coligan, et al., Unit 9, Current Protocols In Immunology, Wiley Interscience, 1991, incorporated by reference.) One of skill in the art would readily appreciate that monoclonal antibodies specific to MST1 phospho-T120 could also be prepared using conventional methods.

The term “antibody” as used in this invention includes intact molecules as well as fragments thereof, such as Fab, Fab′₂ and Fv, which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of a whole antibody with the enzyme papain to yield an intact light chain and a portion of one heavy chain;
(2) Fab′₂, the fragment of an antibody molecule, can be obtained by treating a whole antibody with pepsin, followed by reduction, to yield an intact light chain and a portion of the heavy chain; two Fab′ fragments are obtained per antibody molecule;
(3) Fab′2, the fragment of the antibody that can be obtained by treating the whole antibody with the enzyme pepsin without subsequent reduction; Fab′₂ is a dimmer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See, for example, Harlow and Lane, Antibodies: A Laboratory Manual, Cold Spring Harbor Laboratory, New York (1988), incorporated herein by reference.)

The antibodies of the present invention can be used to isolate MST1 phospho-T120. Additionally, the antibodies are useful for detecting the presence of MST1 phospho-T120.

Immunological procedures useful for in vitro detection of target MST1 phospho-T120 in a sample include immunoassays that employ a detectable antibody; such immunoassays include, for example, ELISA, Pandex microfluorimetric assay, agglutination assays, flow cytometry, serum diagnostic assays and immunohistochemical staining procedures which are well known in the art. An antibody can be made detectable by various means well-known in the art. For example, a detectable marker can be directly or indirectly attached to the antibody; useful markers include, for example, radionucleotides, enzymes, fluorogens, chromogens and chemiluminescent labels.

In certain embodiments, the invention teaches an isolated antibody that specifically binds to MST1 phospho-T120. In some embodiments, the invention teaches a composition comprising an antibody that specifically binds to MST phospho-T120.

In some embodiments, the invention teaches a method of detecting MST1 phospho-T120 in a sample, including: providing a sample; contacting the sample with an antibody that binds to MST1 phospho-T120; allowing the antibody to bind to MST1 phospho-T120 in the sample; and detecting the presence of MST1 phospho-T120 in the sample. In certain embodiments, the sample is obtained from a tumor. In certain embodiments, the detection is accomplished by immunohystochemical analysis. In certain embodiments, the sample contains cells. In certain embodiments, the sample contains tissue.

In some embodiments, the invention teaches a method of determining late stage cancer in a subject, including: providing a sample of cells from a subject; contacting the sample with an antibody that binds to MST1 phospho-T120; allowing the antibody to bind to MST1 phospho-T120 in the sample; and detecting the presence of MST1 phospho-T120 in the nucleus of the cells and not in the cytoplasm of the cells, wherein detection of MST1 phospho-T120 in the nucleus of the cells and not in the cytoplasm indicates late stage cancer. In certain embodiments, the sample is obtained from a tumor. In some embodiments, the detection is accomplished by immunohystochemical analysis. In some embodiments, the cancer is prostate cancer. In certain embodiments, cell lysates are used.

In various embodiments, the invention teaches a method of determining the amount of MST1 phospho-T120 in the cells of a sample, including: providing a sample of cells from a subject; contacting the sample with an antibody that binds to MST1 phospho-T120; and determining the amount of MST1 phospho-T120 in the cells of the sample. In some embodiments, the sample is obtained from a tumor. In some embodiments, the amount of MST1 phospho-T120 is determined using immunohystochemical analysis. In certain embodiments, cell lysates are used.

In certain embodiments, the invention teaches a method of determining early stage cancer in a subject, including: providing a sample of cells from a subject; contacting the sample with an antibody that binds to MST1 phospho-T120; allowing the antibody to bind to MST1 phospho-T120 in the sample; detecting the presence of MST1 phospho-T120 in the cytoplasm of the cells and in the nucleus of the cells, wherein detection of MST1 phospho-T120 in the cytoplasm of the cells and in the nucleus of the cells indicates early stage cancer in the subject. In certain embodiments, the cancer is prostate cancer. In certain embodiments, cell lysates are used

In certain embodiments, the invention teaches a method of determining the invasiveness of prostate cancer (PCa) in a subject, including: determining that a modification is present in the MST1 protein of the prostate cancer cells, compared to the MST1 protein of non-cancerous cells.

In certain embodiments, the invention teaches a method of screening for compounds that regulate the levels of MST1 phospho-T120, directly or indirectly. In certain embodiments, the levels of MST1 phospho-T120 are determined before and/or after administering a compound to a subject. In certain embodiments, MST1 phospho-T120 levels are determined using the antibodies of the present invention, according to one or more of the inventive methods for visualizing detection described herein. In certain embodiments, the method is performed in vitro.

In certain embodiments, the invention teaches a method of diagnosing prostate cancer in a subject including: providing a sample of cells from a subject; contacting the sample with an antibody that binds to MST1 phospho-T120; allowing the antibody to bind to MST1 phospho-T120 in the sample; and detecting the presence of MST1 phospho-T120 in the cells of the sample, wherein detection of MST1 phospho-T120 in the cytoplasm of the cells and in the nucleus of the cells or only in the nucleus of the cells indicates prostate cancer. In certain embodiments, the sample is obtained from a tumor. In some embodiments, the detection is accomplished by immunohystochemical analysis.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive. One skilled in the art will recognize many methods and materials similar or equivalent to those described herein, which could be used in the practice of the present invention. Indeed, the present invention is in no way limited to the methods and materials described. Various embodiments of the invention are described above in the Description of the Invention. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Example 1

### The Development and Assessment of MST1 Phospho-T120 Antibody Immunogen

The MST1 phospho-T120 antibody was produced in rabbits against the synthesized and T120 phosphorylated peptide of SEQ ID NO: 1 CSDIIRLRNK{pThr}LTEDEIA corresponding to the N terminus (111-127 amino acid) of human MST1 protein. The antibody produced was rabbit polyclonal antibody.

### Example 2

### Specificity

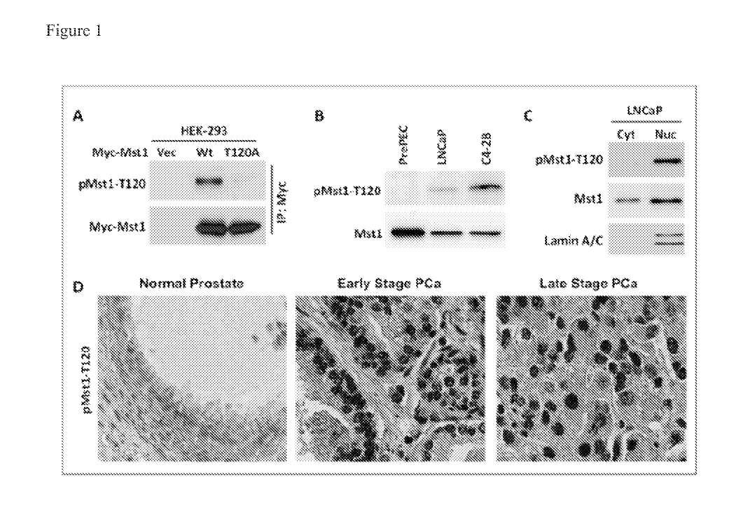

Immunoprecipitation, western blot, and immunohistochemistry experiments were performed in total cell lysates to determine whether the antibody specifically recognizes MST1 phospho-T120 signal in cells and in tissue. Myc-tagged wild type (wt) or phosphorylation-deficient (Thr120Ala) mutant MST1 was transiently expressed in HEK (Human Embryonic Kidney) 293 cells. Empty vector (Vec) was also transfected as a negative control. A threonine to Alanine (T-A) mutation was made using a standard mutagenesis approach. Total MST1-wt and MST1-T120A mutant was immunoprecipitated with anti-Myc antibody from total cell Lysates, and the antibody-protein complexes were resolved by SDS-PAGE. The western blot analysis revealed that phospho-T120 antibody failed to detect the phosphorylation-deficient MST1 when the result is compared to that of the intact MST1 phospho-T120 (FIG. 1A). This indicates that the antibody is specific to MST-T120 site phosphorylation. Second, the levels of MST1 phospho-T120 were determined by western blot in total lysates from primary prostate epithelial cells (PrePEC) and from PCa cells (androgen-sensitive LNCaP line and castration resistant or androgen-insensitive C4-2 or C4-2B line) that were grown under steady state conditions (i.e., serum containing condition). The results shown in FIG. 1B demonstrate that MST1 phosphorylation at T120 site is detectable only in cancer cells with increased levels with progression to castration-resistance state and no detectible levels of MST1 phospho-T120 were observed in normal prostate cells. Third, the inventors prepared nuclear and cytoplasmic extract from LNCaP PCa cells to assess the subcellular localization of MST1 phospho-T120 in the cell. Western blot analysis revealed that Mst1-T120 site phosphorylation appears to occur only in cell nuclei because no detectable Mst1 phospho-T120 was observed in the cytoplasmic fraction of LNCaP cells that were grown under serum-depleted conditions (FIG. 1C). Finally, the inventors conducted an immunohystochemical analyses of MST1 phospho-T120 levels in formalin-fixed and paraffin-embedded sections, which were prepared from the normal prostate and early or late stage human PCa tissues. The results of this experiment showed that MST1 phospho-T120 was primarily enriched in the cytoplasm in normal tissue; however, MST1 phospho-T120 can be seen both in the cytoplasm and in the nucleus of cell in the early stage of PCa and the phospho-T120 was detected only in cell nuclei of cells in the late stage of PCa (FIG. 1D).

What is claimed is:

1. An isolated antibody that specifically binds to MST1 phospho-T120.

2. A composition comprising an antibody that specifically binds to MST phospho-T120.

3. A method of detecting MST1 phospho-T120 in a sample comprising:
(a) providing a sample;
(b) contacting the sample with an antibody that binds to MST1 phospho-T120;
(c) allowing the antibody to bind to MST1 phospho-T120 in the sample; and
(d) detecting the presence of MST1 phospho-T120 in the sample.

4. The method according to claim 3, wherein the sample is obtained from a tumor.

5. The method according to claim 3, wherein the detection is accomplished by immunohystochemical analysis.

6. A method of determining late stage cancer in a subject comprising:
(a) providing a sample of cells from a subject;
(b) contacting the sample from the subject with an antibody that binds to MST1 phospho-T120;
(c) allowing the antibody to bind to MST1 phospho-T120 in the sample; and
(d) detecting the presence of MST1 phospho-T120 in the nucleus of the cells and not in the cytoplasm of the cells, wherein detection of MST1 phospho-T120 in the nucleus of the cells and not in the cytoplasm indicates late stage cancer.

7. The method according to claim 6, wherein the sample is obtained from a tumor.

8. The method according to claim 6, wherein the detection is accomplished by using immunohystochemical analysis.

9. The method according to claim 6, wherein the cancer is prostate cancer.

10. A method of determining the amount of MST1 phospho-T120 in the cells of a sample, comprising:
(a) providing a sample of cells from a subject;
(b) contacting the sample with an antibody that binds to MST1 phospho-T120; and
(c) determining the amount of MST1 phospho-T120 in the cells of the sample.

11. The method according to claim 10, wherein the sample is obtained from a tumor.

12. The method according to claim 10, wherein the amount of MST1 phospho-T120 is determined using immunohystochemical analysis.

13. A method of determining early stage cancer in a subject comprising:
(a) providing a sample of cells from a subject;
(b) contacting the sample with an antibody that binds to MST1 phospho-T120;
(c) allowing the antibody to bind to MST1 phospho-T120 in sample;
(d) detecting the presence of MST1 phospho-T120 in the cytoplasm of the cells and in the nucleus of the cells, wherein detection of MST1 phospho-T120 in the cytoplasm of the cells and in the nucleus of the cells indicates early stage cancer in the subject.

14. A method of determining the invasiveness of prostate cancer (PCa) in a subject, comprising determining that a modification is present in the MST1 protein of the prostate cancer cells, compared to the MST1 protein of non-cancerous cells.

15. A method of diagnosing prostate cancer in a subject comprising:
(a) providing a sample of cells from a subject;
(b) contacting the sample from the subject with an antibody that binds to MST1 phospho-T120;
(c) allowing the antibody to bind to MST1 phospho-T120 of the sample; and
(d) detecting the presence of MST1 phospho-T120 in the cells of the sample, wherein detection of MST1 phospho-T120 in the cytoplasm of the cells and in the nucleus of the cells or only in the nucleus of the cells indicates prostate cancer.

16. The method according to claim 15, wherein the sample is obtained from a tumor.

17. The method according to claim 15, wherein the detection is accomplished by immunohystochemical analyses.


SEQUENCE LISTING (1 sequence)

SEQ ID NO: 1 - protein, 18 aa, Homo sapiens
CSDIIRLRNKTLTEDEIA